(19) United States
(12) Patent Application Publication
(10) Pub. No.: US 2006/0193789 A1
(43) Pub. Date: Aug. 31, 2006

(54) Film forming foamable composition

(75) Inventors: Dov Tamarkin, Maccabim (IL); Doron Friedman, Karmei Yosef (IL); Meir Eini, Ness Ziona (IL)

Correspondence Address: WILMER CUTLER PICKERING HALE AND DORR LLP, 60 STATE STREET, BOSTON, MA, 02109, US

(73) Assignee: Foamix Ltd., Rehovot (IL)

(21) Appl. No.: 11/337,747

(22) Filed: Jan. 23, 2006

Related U.S. Application Data
(63) Continuation-in-part of application No. 10/911,367, filed on Aug. 4, 2004.
(63) Continuation-in-part of application No. PCT/IB03/05527, filed on Oct. 24, 2003.
(63) Continuation-in-part of application No. 10/922,358, filed on Aug. 20, 2004.
(60) Provisional application No. 60/492,385, filed on Aug. 4, 2003.
(60) Provisional application No. 60/429,546, filed on Nov. 29, 2002.
(60) Provisional application No. 60/497,648, filed on Aug. 25, 2003.

(30) Foreign Application Priority Data
Oct. 25, 2002 (IL) 152486

(51) Int. Cl. A61K 8/73

(52) U.S. Cl. 424/47; 424/70.13

(57) ABSTRACT

A foamable composition, includes (1) about 6% to about 70% by weight of at least one organic carrier; (2) about 0.1% to about 5% by weight of at least one surface-active agent; (3) about 0.01% to about 5% by weight of at least one film forming agent; (4) water; and (5) about 3% to about 25% by weight of the total composition of at least one liquefied or compressed gas propellant. The composition is substantially alcohol free and is used in treating, alleviating or preventing a disorder.

### CROSS REFERENCE TO RELATED APPLICATIONS

This application is a continuation-in-part application of co-pending International Patent Application No. IB03/005527, designating the United States and filed on Oct. 24, 2003, which claims the benefit of priority under 35 U.S.C. §119(e) to U.S. Patent Application Ser. No. 60/429,546, filed on Nov. 29, 2002, both entitled “Cosmetic and Pharmaceutical Foam,” and which claims the benefit of priority under 35 USC§119(a) to Israeli Patent Application No. 152486, filed Oct. 25, 2002, all of which are hereby incorporated in their entirety by reference.

This application is a continuation-in-part application of co-pending U.S. patent application Ser. No. 10/911,367, filed on Aug. 4, 2004, which claims the benefit of priority under 35 U.S.C. §119(e) to U.S. Patent Application Ser. No. 60/492,385, filed on Aug. 4, 2003, both entitled “Foam Carrier Containing Amphiphilic Copolymer Gelling Agent” and both hereby incorporated in their entirety by reference.

This application is a continuation-in-part application of co-pending U.S. patent application Ser. No. 10/922,358, filed Aug. 20, 2004, entitled “Penetrating Pharmaceutical Foam,” which claims the benefit of priority under 35 U.S.C. §119(e) to U.S. Patent Application Ser. No. 60/497,648, filed on Aug. 25, 2003, and which claims the benefit of priority under 35 USC§119(a) to Israeli Patent Application No. 152486, filed Oct. 25, 2002, all of which are incorporated by reference.

### BACKGROUND OF THE INVENTION

This invention relates to alcohol-free foamable pharmaceutical and cosmetic compositions that possesses a film-forming property.

Foams and, in particular, foam emulsions are complicated systems which do not form under all circumstances. Changes in foam emulsion composition, such as by the addition of active ingredients may destabilize the foam.

U.S. Pat. No. 6,126,920 discloses treatment of various skin diseases, and in particular, scalp psoriasis, using a foamable pharmaceutical composition containing a corticosteroid active substance, an aliphatic alcohol, water, a fatty alcohol, a surface-active agent, a propellant and a buffering agent. The foamable composition contains 40-90% w/w composition of an aliphatic alcohol. U.S. Pat. No. 6,126,920 is typical of many compositions that use aliphatic alcohols in the foam composition. The alcohol promotes fast drying and thereby attempts to address the sticky feeling left by many topical formulations after application; however, alcohols, and in particular the methyl, ethyl and isopropyl alcohols preferred in the '920 patent, are defatting agents and may cause skin to become dry and cracked.

US Published Application No. 2004/0151671 provides pharmaceutical compositions in a pressurized container, comprising a quick breaking alcoholic foaming agent.

U.S. Pat. No. 5,783,202 provides a pediculicidal mousse composition containing a pediculicidal agent containing (a) from about 0.1 to about 10% w/w of a pediculicidal agent (b) about 70 to about 97% w/w of a foaming agent, which is preferably a quick breaking alcoholic foaming agent; and (c) from about 3 to about 20% w/w of an aerosol propellant.

U.S. Pat. No. 6,730,288 teaches a pharmaceutical foam composition including (a) an active ingredient; (b) an occlusive agent; (c) an aqueous solvent; and (d) an organic cosolvent; wherein the active ingredient is insoluble in water and insoluble in both water and the occlusive agent; and wherein there is enough occlusive agent to form an occlusive layer on the skin.

WO 2004/071479 describes a composition in a pressurized container for forming a hydroalcoholic foam, comprising at least one active agent, a water insoluble film forming polymer, a hydroalcoholic foaming agent and an aerosol propellant.

### BRIEF DESCRIPTION OF THE INVENTION

The present invention provides a film-forming foamable cosmetic or pharmaceutical vehicle, and cosmetic and/or pharmaceutical compositions thereof.

In one or more embodiments, the foamable cosmetic or pharmaceutical vehicle includes:
(1) about 6% to about 70% by weight of an organic carrier;
(2) about 0.1% to about 5% by weight of a surface-active agent;
(3) about 0.01% to about 5% by weight of a film forming agent; and
(4) about 3% to about 25% by weight of the total composition of a liquefied or compressed gas propellant.

Water and optional ingredients are added to complete the total mass to 100%.

In one or more embodiments, a pharmaceutical or cosmetic foamable product is provided, wherein a pharmaceutical or a cosmetic active agent is incorporated in a foamable vehicle, which contains a polar solvent and an hydrophobic organic carrier

Thus, in one or more embodiments, the pharmaceutical or cosmetic foamable product includes:
(1) an effective concentration of a pharmaceutical or cosmetic active agent;
(2) an organic carrier, at a concentration of about 6% to about 70% by weight;
(3) about 0.1% to about 5% by weight of a surface-active agent; and
(4) a liquefied or compressed gas propellant at a concentration of about 3% to about 25% by weight of the total composition.

Water and optional ingredients are added to complete the total mass to 100%.

All % values are provided on a weight (w/w) basis.

### DETAILED DESCRIPTION OF THE INVENTION

The foamable composition is substantially alcohol-free, i.e., free of short chain monohydric alcohols. Short chain monohydric alcohols, having up to 5 carbon atoms in their carbon chain skeleton and one hydroxyl group, such as ethanol, propanol, isopropanol, butanol, iso-butanol, t-butanol and pentanol, are considered less desirable solvents or polar solvents due to their skin-irritating effect. Thus, the composition is substantially alcohol-free and includes less than about 5% final concentration of lower alcohols, preferably less than about 2%, more preferably less than about 1%.

The foamable composition of the present invention can be an emulsion, or microemulsion, including an aqueous phase and an organic carrier phase. The organic carrier, also termed herein interchangeably “organic solvent” or “solvent”, is selected from a hydrophobic organic carrier (also termed herein “hydrophobic solvent”), an emollient, a polar solvent, and a mixture thereof.

Organic Carrier

The organic carrier includes hydrophobic solvent carriers, polar solvent carriers and emollients, and mixtures thereof. The identification of an organic carrier (or “solvent”), as used herein, is not intended to characterize the solubilization capabilities of the carrier for any specific active agent or any other component of the foamable composition. Rather, such information is provided to aid in the identification of materials suitable for use as an organic carrier in the foamable compositions described herein. A “hydrophobic organic carrier” as used herein refers to a material having solubility in distilled water at ambient temperature of less than about 1 gm per 100 mL, more preferable less than about 0.5 gm per 100 mL, and most preferably less than about 0.1 gm per 100 mL. It is liquid at ambient temperature.

In one or more embodiments, the hydrophobic organic carrier is an oil, such as mineral oil. Mineral oil (Chemical Abstracts Service Registry number 8012-95-1) is a mixture of aliphatic, naphthalenic, and aromatic liquid hydrocarbons that derive from petroleum. It is typically liquid; its viscosity is in the range of between about 35 CST and about 100 CST (at 40° C.), and its pour point (the lowest temperature at which an oil can be handled without excessive amounts of wax crystals forming so preventing flow) is below 0° C.

According to one or more embodiments, hydrophobic solvents are liquid oils originating from vegetable, marine or animal sources. Suitable liquid oil includes saturated, unsaturated or polyunsaturated oils. By way of example, the unsaturated oil may be olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils or mixtures thereof, in any proportion.

Suitable hydrophobic solvents also include polyunsaturated oils containing polyunsaturated fatty acids. In one or more embodiments, the unsaturated fatty acids are selected from the group of omega-3 and omega-6 fatty acids. Examples of such polyunsaturated fatty acids are linoleic and linolenic acid, gamma-linoleic acid (GLA), eicosapentaenoic acid (EPA) and docosahexaenoic acid (DHA). Such unsaturated fatty acids are known for their skin-conditioning effect, which contribute to the therapeutic benefit of the present foamable composition. Thus, the hydrophobic solvent can include at least 6% of an oil selected from omega-3 oil, omega-6 oil, and mixtures thereof. In the context of the present invention, oils that possess therapeutically beneficial properties are termed “therapeutically active oil”.

Another class of hydrophobic solvents is the essential oils, which are also considered therapeutically active oil, which contain active biologically occurring molecules and, upon topical application, exert a therapeutic effect, which is conceivably synergistic to the beneficial effect of the steroid in the composition.

Another class of therapeutically active oils includes liquid hydrophobic plant-derived oils, which are known to possess therapeutic benefits when applied topically.

Silicone oils also may be used and are desirable due to their known skin protective and occlusive properties. Suitable silicone oils include non-volatile silicones, such as polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. These are chosen from cyclic or linear polydimethylsiloxanes containing from about 3 to about 9, preferably from about 4 to about 5, silicon atoms. Volatile silicones such as cyclomethicones can also be used. Silicone oils are also considered therapeutically active oil, due to their barrier retaining and protective properties.

In one or more embodiments, the hydrophobic carrier includes at least 2% by weight silicone oil or at least 5% by weight.

The solvent may be a mixture of two or more of the above hydrophobic solvents in any proportion.

A further class of organic carrier includes “emollients” that have a softening or soothing effect, especially when applied to body areas, such as the skin and mucosal surfaces. Emollients are not necessarily hydrophobic. Examples of suitable emollients include hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate and mixtures thereof.

According to one or more embodiments of the present invention, the hydrophobic organic carrier includes a mixture of a hydrophobic solvent and an emollient. According to one or more embodiments, the foamable composition is a mixture of mineral oil and an emollient in a ratio between 2:8 and 8:2 on a weight basis.

A “polar solvent” is an organic solvent, typically soluble in both water and oil. Examples of polar solvents include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, sulfoxides, such as dimethylsulfoxide (DMSO), dimethylformanide, methyl dodecyl sulfoxide, dimethylacetamide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one or more embodiments, the polar solvent is a polyethylene glycol (PEG) or PEG derivative that is liquid at ambient temperature, including PEG200 (MW (molecular weight) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

In one or more embodiments, a pharmaceutically or cosmetically active agent is an organic substance which is semi-liquid or liquid at ambient temperature. As such, a semi-liquid or liquid pharmaceutically or cosmetically active agent can be considered as part of the “organic carrier”, as defined herein. Non-limiting examples of semi-liquid or liquid pharmaceutically or cosmetically active agent include, but are not limited to permethrin, pyrethrum extract, piperonyl butoxide, diethyl toluamide (DEET), dimethyl phthalate and vitamin E (tocopherol). Several sunscreen agents are semi-liquid or liquid. The semi-liquid or liquid pharmaceutically or cosmetically active agent can be included in the foamable composition as a sole organic carrier component, or in combination with additional organic carriers, as detailed above. In certain case, such as the DEET composition for insect repellency the only “organic carrier” is DEET, which is present at a 5%-30% concentration and more preferably at a concentration of 15-20%.

In certain embodiments, the organic carrier is a combination of a hydrophobic carrier and a polar solvent; or a combination of an emollient and a polar solvent; or a combination of a hydrophobic carrier, an emollient and a polar solvent. Polar solvents may be added to the foam composition for a variety of reasons, such as to increase drug solubilization, promote dermal drug delivery or provide a desired sensory feeling. However, polar solvents tend to dry the skin and impair the integrity of the skin barrier. By contrast, by combining a polar solvent and a hydrophobic carrier, or an emollient or both, unwanted skin barrier damage is reduced. A ratio of 8:1 and 1:4 between the hydrophobic carrier/emollient and the polar solvent is preferred.

Film Forming Agent

The foamable composition of the present invention contains a film forming agent. The film forming agent serves to stabilize the foam composition and to control drug residence in the target organ, thereby limiting the rate of its clearance from the site. The film formation properties of the foamable composition help maintain active ingredients on the site of application by imparting resistance to abrasion or rub-off. Furthermore, in certain cases, the film that is formed promotes the penetration of active agents into the skin (intradermal penetration) and through the skin (transdermal penetration), as it creates an occlusive or semi-occlusive layer at the treatment site.

The film forming component may include at least one water-insoluble alkyl cellulose or hydroxyalkyl cellulose. Exemplary alkyl cellulose or hydroxyalkyl cellulose polymers include ethyl cellulose, propyl cellulose, butyl cellulose, cellulose acetate, hydroxypropyl cellulose, hydroxybutyl cellulose, and ethylhydroxyethyl cellulose, alone or in combination. In addition, a plasticizer or a cross linking agent may be used to modify the polymer's characteristics. For example, esters such as dibutyl or diethyl phthalate, amides such as diethyldiphenyl urea, vegetable oils, fatty acids and alcohols such as oleic and myristyl acid may be used in combination with the cellulose derivative.

Exemplary commercially available film forming agent is DERMACRYL from National Starch and Chemical Co. These hydrophobic, high-molecular-weight, carboxylated acrylic copolymers are compatible with a broad range of cosmetic raw materials. They can be used in traditional O/W and W/O emulsions. Exemplary DERMACRYL copolymers, suitable as film forming agents according to the present invention include DERMACRYL-LT, DERMACRYL-79 and DERMACRYL-AQF.

Another exemplary commercially available film forming agent is Aculyn from Rohm & Haas. Aculyn is an anionic hydrophobically modified alkali-soluble acrylic polymer emulsion (HASE) this is lightly crosslinked with unusually high aqueous thickening and stabilizing efficiency. Suitable acrylate copolymers are exemplified by, but are not limited to Aculyn 22 (acrylates/steareth-20 methacrylate copolymer—Rohm & Haas) Aculyn 25 (acrylates/laureth-25 methacrylate copolymer—Rohm & Haas), Aculyn 28 (acrylates/beheneth-25 methacrylate copolymer—Rohm & Haas), Aculyn 46 (PRG-150/stearyl alcohol/SMDI copolymer—Rohm & Haas). Analogous acrylate copolymers from alternative commercial sources are also suitable.

Additional non-limiting examples of film forming polymers include, but are not limited to hydrophobically-modified polysaccharides (such as Natrosol Plus from Hercules), hydrophobically-modified ethoxylated urethanes (such as the RM series from Rohm & Haas), Acrylates/Alkyl Acrylate Cross-Polymer (such as Pemulen TR-1 and Pemulen TR-2), Carbopol 1382 (Acrylates/C10-30 alkyl acrylate crosspolymer—Noveon), Natrosol CS Plus 330, 430, Polysurf 67 (cetyl hydroxyethyl cellulose—Hercules), Stabylen 30 (acrylates/vinyl isodecanoate—3V), Structure 2001 (acrylates/steareth-20 itaconate copolymer—National Starch), Structure 3001 (acrylates/ceteth-20 itaconate copolymer—National Starch), and Structure Plus (acrylates/aminoacrylates/C10-30 alkyl PEG 20 itaconate copolymer—National Starch), polyvinylpyrrolidone/vinylacetate (Luviskol VA 73W from BASF), polyurethane-1 (Luvi-set PUR from BASF or PUR 28-001A from National Starch), polyvinylcaprolactam (Luvitec VCAP from BASF), polyvinylpyrrolidone/polyvinylcaprolactam (Luvitec VPC from BASF), polyvinylpyrrolidone/dimethylaminopropylmethacrylamide (Styleze CC-10 from ISP), polyvinylpyrrolidone/polyvinylcaprolactam/dimethylaminopropylmethacrylamide (Aquaflex SF-40 from ISP), isobutylene ethylmaleimide/hydroxyethylmaleimide (Aquaflex FX-64 from ISP), polyvinyl-pyrrolidone/dimethylaminoethylmeth-acrylate (Copolymer 845 from ISP), quaternized polyvinyl-pyrrolidone/di-methylaminoethylmethacrylate (Gafquat 734, Gafquat 755, or Gafquat 755N from IS P), polyvinylpyrrolidone/polyvinylcaprolactam/dimethyl-aminoethylmethacrylate (Gaffix VC-713 from ISP), and poly(vinylacetate/crotonates/vinylneodecanoate) (Resyn 28-2930 from National Starch) and octylacrylamide/acrylate/butylamino-ethylmethacrylate (Amphomer from National Starch).

In one or more embodiments, the film forming agent is chitosan. Chitosan is a natural polymer obtained by the hydrolysis of chitin, a native polymer present in shellfish. Together with chitin, chitosan is considered the second most abundant polysaccharide after cellulose. Chitosan forms a film by binding to lipids, proteins and many other biologically active substances of the skin, hair and other tissues. Importantly, in addition to its film-forming property, chitosan has antiinfective properties. For example, chitosan is known to inhibit the adhesion of candida albicans to human cells and tissues.

In one or more embodiments, the film forming agent is pullulan. Pullulan is a polysaccharide produced from a cultivated fungus of Aureobasidium pullulans (pulluaria pullulans). Pullulan is an alpha-glucan mainly constituted of maltrotriose as repeating units linearly joined through alpha-1,6-glycosidic linkages, not branched. Preferably, the pullulan used in the compositions of the invention will exhibit a molecular weight of 200,000 to 2,000,000 Daltons. In certain embodiments, the film forming agent is a combination of pullulan and polyvinyl alcohol, wherein the composition contains 3 to 30% pullulan and 3 to 30 wt % polyvinyl alcohol.

The film forming agent is present in an amount in the range of about 0.01% to about 5.0% by weight of the foam composition. In one or more embodiments, it is typically less than about 1 wt % of the foamable composition.

Typically, due to their polymeric nature and their tendency to form polymeric aggregate and/or networks, film forming agents also possess gelling properties.

In order to derive a composition which is readily foamable upon release from a pressurized container, additional components are required, as provided hereinbelow.

Surface-active agents (also termed “surfactants”) include any agent linking oil and water in the composition, in the form of emulsion. A surfactant's hydrophilic/lipophilic balance (HLB) describes the emulsifier's affinity toward water or oil. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average). The surface active agent according to the present invention has an HLB value, suitable for stabilizing an emulsion comprising the aqueous phase and the organic carrier of the composition.

According to one or more embodiments of the present invention, the surface-active agent has a hydrophilic lipophilic balance (HLB) between about 9 and about 14, which is the required HLB (the HLB required to stabilize an O/W emulsion of a given oil) of most oils and hydrophobic solvents. Thus, in one or more embodiments, the composition contains a single surface active agent having an HLB value between about 9 and 14, and in one or more embodiments, the composition contains more than one surface active agent and the weighted average of their HLB values is between about 9 and about 14. Yet, in other embodiments, when a water in oil emulsion is desirable, the composition contains one or more surface active agents, having an HLB value between about 2 and about 9.

The surface-active agent is selected from anionic, cationic, nonionic, zwitterionic, amphoteric and ampholytic surfactants, as well as mixtures of these surfactants. Such surfactants are well known to those skilled in the therapeutic and cosmetic formulation art. Non-limiting examples of possible non-ionic surfactants include polysorbates, such as polyoxyethylene (20) sorbitan monostearate (Tween 60) and poly(oxyethylene) (20) sorbitan monooleate (Tween 80); poly(oxyethylene) (POE) fatty acid esters, such as Myrj 45, Myrj 49, Myrj 52 and Myrj 59; poly(oxyethylene)alkylyl ethers, such as poly(oxyethylene)cetyl ether, poly(oxyethylene)palmityl ether, polyethylene oxide hexadecyl ether, polyethylene glycol cetyl ether, brij 38, brij 52, brij 56 and brij W1; sucrose esters, partial esters of sorbitol and its anhydrides, such as sorbitan monolaurate and sorbitan monolaurate; mono or diglycerides and isoceteth-20

Surfactants such as sodium methyl cocoyl taurate, sodium methyl oleoyl taurate, sodium lauryl sulfate, triethanolamine lauryl sulfate and betaines are ionic, i.e., anionic, cationic or zwitterionic.

In one or more embodiments of the present invention, the surface-active agent includes an non-ionic surfactant. Ionic surfactants are known to be effective as foaming agents, however, they are also known for their skin and mucosal irritancy. Therefore, non-ionic surfactants are preferred in applications including sensitive tissue such as found in most mucosal tissues, especially when they are infected or inflamed. We have surprisingly found that non-ionic surfactants alone, which have a lesser foaming effect provide foams of excellent quality, i.e. a foam quality score “E” according to the grading scale discussed hereinbelow.

In one or more embodiments, the surface active agent includes a mixture of an non-ionic surfactant and an ionic surfactant in a ratio in the range of about 100:1 to 6:1. In one or more embodiments, the non-ionic to ionic surfactant ratio is greater than about 6:1, or greater than about 8:1; or greater than about 14:1, or greater than about 16:1, or greater than about 20:1.

In one or more embodiments of the present invention, a combination of a non-ionic surfactant and an ionic surfactant (such as sodium lauryl sulphate and cocamidopropylbetaine) is employed, at a ratio of between 1:1 and 20:1, or at a ratio of 4:1 to 10:1. The resultant foam has a low specific gravity, e.g., less than 0.1 g/ml.

Thus, in an exemplary embodiment, a combination of an non-ionic surfactant having HLB of less than 9 and an non-ionic surfactant having HLB of equal or more than 9 is employed, at a ratio of between 1:8 and 8:1, or at a ratio of 4:1 to 1:4, wherein the HLB of the combination of emulsifiers is between about 9 and about 14.

In one or more embodiments of the present invention, the surface-active agent includes mono-, di- and tri-esters of sucrose with fatty acids (sucrose esters), prepared from sucrose and esters of fatty acids or by extraction from sucro-glycerides. Suitable sucrose esters include those having high monoester content, which have higher HLB values.

The total surface active agent is in the range of about 0.1 to about 5% of the composition, and is occasionally less than about 2% or less than about 1%.

Optionally, gelling agents can be included in the composition of the present invention, in addition to the film forming agent. Gelling agents or other polymeric additive may be present in a range of about 0.01 wt % to about 5 wt %. Exemplary gelling agents that can be optionally used include, but are not limited to, naturally-occurring polymeric materials such as, locust bean gum, sodium alginate, sodium caseinate, egg albumin, gelatin agar, carrageenin gum sodium alginate, xanthan gum, quince seed extract, tragacanth gum, starch, chemically modified starches and the like, semi-synthetic polymeric materials such as cellulose ethers (e.g. hydroxyethyl cellulose, methyl cellulose, carboxymethyl cellulose, hydroxy propylmethyl cellulose), polyvinylpyrrolidone, polyvinylalcohol, guar gum, hydroxypropyl guar gum, soluble starch, cationic celluloses, cationic guars and the like and synthetic polymeric materials such as carboxyvinyl polymers, polyvinylpyrrolidone, polyvinyl alcohol polyacrylic acid polymers, polymethacrylic acid polymers, polyvinyl acetate polymers, polyvinyl chloride polymers, polyvinylidene chloride polymers and the like. Mixtures of the above compounds are contemplated.

Further exemplary gelling agents include the acrylic acid/ethyl acrylate copolymers and the carboxyvinyl polymers, which consist essentially of a colloidal water-soluble polyalkenyl polyether crosslinked polymer of acrylic acid crosslinked with a crosslinking agent such as polyallyl sucrose or polyallyl pentaerythritol. Examples include Carbopol® 934, Carbopol® 940, Carbopol® 950, Carbopol® 980, Carbopol® 951 and Carbopol® 981.

In one or more embodiments, the gelling agent is a phase change polymer, which alters the composition behavior from fluid-like prior to administration to solid-like upon contact with the target mucosal surface. Such phase change results from external stimuli, such as changes in temperature or pH and exposure to specific ions (e.g., Ca²⁺). Non-limiting examples of phase change polymers include poly(N-isopropylamide) and Poloxamer 407®.

Yet, in other embodiments, the gelling agent includes inorganic gelling agents, such as silicone dioxide (fumed silica).

A further class of gelling agents includes mucoadhesive/bioadhesive polymers in an amount sufficient to confer bioadhesive properties. The bioadhesive polymers can be selected from acidic synthetic polymers, preferably having an acidic group per four repeating or monomeric subunit moieties, such as poly(acrylic)- and/or poly(methacrylic) acid (e.g., Carbopol®, Carbomer®), poly(methylvinyl ether/maleic anhydride) copolymer, and their mixtures and copolymers; acidic synthetically modified natural polymers, such as carboxymethylcellulose (CMC); neutral synthetically modified natural polymers, such as (hydroxypropyl)methylcellulose; basic amine-bearing polymers such as chitosan; acidic polymers obtainable from natural sources, such as alginic acid, hyaluronic acid, pectin, gum tragacanth, and karaya gum; and neutral synthetic polymers, such as polyvinyl alcohol or their mixtures. An additional group of mucoadhesive polymers includes natural and chemically modified cyclodextrin, especially hydroxypropyl-β-cyclodextrin. Such polymers may be present as free acids, bases, or salts, usually in a final concentration of about 0.01% to about 0.5% by weight. Many mucoadhesive agents are known in the art to also possess gelling properties.

Mixtures of gelling agents are contemplated.

Optionally, a foam adjuvant is included in the foamable compositions of the present invention to increase the foaming capacity of surfactants and/or to stabilize the foam. Foam adjuvants may be present in a range of about 0.1 wt % to about 5 wt %. In one or more embodiments of the present invention, the foam adjuvant agent includes fatty alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1-triacontanol (C30), as well as alcohols with longer carbon chains (up to C50). Fatty alcohols, derived from beeswax and including a mixture of alcohols, a majority of which has at least 20 carbon atoms in their carbon chain, are especially well suited as foam adjuvant agents. The amount of fatty alcohol required to support the foam system is inversely related to the length of its carbon chains. Foam adjuvants, as defined herein are also useful in facilitating improved spreadability and absorption of the composition.

In one or more embodiments of the present invention, the foam adjuvant agent includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. As for fatty alcohols, the amount of fatty acids required to support the foam system is inversely related to the length of its carbon chain.

In one or more embodiments, a combination of a fatty acid and a fatty alcohol is employed. The fatty acids and fatty alcohol may be present in equal amounts, or the proportions may range from 4:1 and 1:4 fatty acid to fatty alcohol

The carbon atom chain of the fatty alcohol or the fatty acid may optionally have one or more double bonds. A further class of foam adjuvant agent includes a branched fatty alcohol or fatty acid. The carbon chain of the fatty acid or fatty alcohol also can be substituted with a hydroxyl group, such as 12-hydroxy stearic acid.

A property of the fatty alcohols and fatty acids used in context of the composition of the present invention is related to their therapeutic properties per se. Long chain saturated and mono unsaturated fatty alcohols, e.g., stearyl alcohol, erucyl alcohol, arachidyl alcohol and behenyl alcohol (docosanol) have been reported to possess antiviral, antiinfective, antiproliferative and antiinflammatory properties (see, U.S. Pat. No. 4,874,794). Longer chain fatty alcohols, e.g., tetracosanol, hexacosanol, heptacosanol, octacosanol, triacontanol, etc., are also known for their metabolism modifying properties and tissue energizing properties. Long chain fatty acids have also been reported to possess anti-infective characteristics.

Active Agent

In one or more embodiments, the foamable carrier of the present invention contains a safe an effective concentration of a pharmaceutical or a cosmetic therapeutic agent (collectively termed herein as “active agent”). The active agent is present in an amount to be effective for its intended purpose. The actual amount may vary widely. For example, in some embodiments, only a few weight percent or even a fraction of a weight percent of active agent is sufficient. In other embodiments, e.g., sun screens and insect repellant, the active agent may constitute a significant component of the foamable composition.

It is to be understood that the active agents useful herein can in some instances provide more than one benefit or operate via more than one mode of action. Therefore, classifications herein are made for the sake of convenience and are not intended to limit the active agent to that particular application or applications listed.

The composition of the present invention comprises an active agent that provides therapeutic or cosmetic activity.

Non-limiting examples of families of active agents include an anti-infective, an antibiotic, an antibacterial agent, a disinfectant, an antifungal agent, an antiviral agent, an antiparasitic agent, an anti-inflammatory agent, an anti-allergic agent, a steroid, a steroidal anti-inflammatory agent, a nonsteroidal anti-inflammatory drug, an immunosuppressive agent, an immunomodulator, an immunoregulating agent, a hormone, a keratolytically active agent, an alpha hydroxyl acid, a beta-hydroxy acid, vitamin A, a vitamin A derivative, a retinoid, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a burn healing agent, an anesthetic, a protein, a peptide, a neuropeptide, a allergen, an immunogenic substance, a haptene, an oxidizing agent, an antioxidant, a dicarboxylic acid, an antiproliferative agent, an anticancer agent, a photodynamic therapy agent, a sunscreen agent, an anti-wrinkle agent, a radical scavenger, a metal oxide, silicone oxide, talc, an anti wrinkle agent, an anti-acne agent, a skin whitening agent, a self tanning agent, an anti-cellulite agent, a skin protective agent, a masking agent, an anti-wart agent, a refatting agent, a lubricating agent and mixtures thereof at any proportion. The concentration of the active agent can be adapted to exert a therapeutic effect on a disease when applied to an afflicted area.

An anti-Infective Agent, selected from an antibiotic agent, an antibacterial agent, an anti-fungal agent, an anti-viral agent and an anti-parasite agent.

An antibiotic or an antibacterial drug can be active against gram positive and gram-negative bacteria, protozoa, aerobic bacteria and unaerobic ones.

In one or more embodiments, the antibiotic agent is selected from the classes consisting of beta-lactam antibiotics, synthetic and semi-synthetic penicillins, aminoglycosides, ansa-type antibiotics, anthraquinones, antibiotic azoles, antibiotic glycopeptides, macrolides, antibiotic nucleosides, antibiotic peptides, antibiotic polyenes, antibiotic polyethers, quinolones, fluoroquinolnes, antibiotic steroids, cyclosporines, sulfonamides, tetracycline, chloramphenicol, dicarboxylic acids, such as azelaic acid, salicylates, antibiotic metals, oxidizing agents, substances that release free radicals and/or active oxygen, cationic antimicrobial agents, quaternary ammonium compounds, biguanides, triguanides, bisbiguanides and analogs and polymers thereof and naturally occurring antibiotic compounds.

Additional antibiotic/antibacterial agents, which is non-specific, include strong oxidants and free radical liberating compounds, such as hydrogen peroxide, bleaching agents (e.g., sodium, calcium or magnesium hypochloride and the like) iodine, chlorohexidine and benzoyl peroxide.

The antifungal agent can be an azole compound. Exemplary azole compounds include azole is an imidazole or triazole selected from the group consisting of azoles, diazoles, triazoles, miconazole, ketoconazole, clotrimazole, econazole, mebendazole, bifonazole, butoconazole, fenticonazole, isoconazole, oxiconazole, sertaconazole, sulconazole, thiabendazole, tiaconazole, fluconazole, itraconazole, ravuconazole and posaconazole.

Additional exemplary antifungal agents include griseofulvin, ciclopirox, amorolfine, terbinafine, Amphotericin B, potassium iodide, flucytosine (5FC) and any combination thereof at a therapeutically effective concentration.

Any known antiviral agent, in a therapeutically effective concentration, can be incorporated in the foam composition of the present invention. Exemplary antiviral agents include, but are not limited to acyclovir, famciclovir, gancyclovir, valganciclovir and abacavir.

According to another embodiment according to the present invention the active agent is an anti-inflammatory or anti-allergic agent. Anti-inflammatory agents can be selected from the group consisting of corticosteroids (also broadly termed herein “steroids”), non-steroidal anti-inflammatory drugs (NSAIDs), anti-histamines, immunosuppressive agents, immunomodulators, immunoregulating agents, and any combination thereof at a therapeutically effective concentration.

Non-limiting examples of corticosteroids include hydrocortisone, hydrocortisone acetate, desonide, Betamethasone, betamethasone valerate, betamethasone dipropionate, betamethasone-17-benzoate, clobetasone-17-butyrate, flucinonide, fluocinolone acetonide, alcometasone dipropionate, mometasone furoate, prednicarbate, triamcinolone acetonide, methylprednisolone aceponate, halcinonide, triamcinolone acetonide, halobetasol and clobetasol-17-propionate.

A second class of anti-inflammatory agents, which is useful in the foam of the present invention, includes the nonsteroidal anti-inflammatory agents (NSAIDs). The variety of compounds encompassed by this group is well-known to those skilled in the art. Specific non-steroidal anti-inflammatory agents useful in the composition invention include, but are not limited to oxicams, such as piroxicam, isoxicam, tenoxicam, sudoxicam; salicylates, such as salicylic acid, ethyl salicylate, methyl salycilate, aspirin, disalcid, benorylate, trilisate, safapryn, solprin, diflunisal, and fendosal; scetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and pyrazoles, such as phenylbutazone, oxyphenbutazone, feprazone, azapropazone, and trimethazone.

Any further steroidal and nonsteroidal compounds, having the capacity to prevent, alleviate the symptoms of, treat or cure inflammation processes, are generally included, as anti-inflammatory agents, according to the present invention.

Antiallergic active agents include antihistamine compounds, including, in a non limiting manner, thylenediamines, such as pyrilamine (mepyramine), antazoline and methapyrilene; tripelennamine phenothiazines, such as promethazine, methdilazine and trimeprazine; ethanolamines, such as diphenhydramine, bromodiphenhydramine, carbinoxamine, clemastine, dimenhydrinate, diphenylpyraline, doxylamine and phenyltoxamine; piperazines, such as cyclizine, buclizine, chlorcyclizine, hydroxyzine, meclizine and thiethylperazine; alkylamines, such as brompheniramine, pyrrobutamin, desbrompheniramine, tripolidine, dexchlorpherniramine, chlorpheniramine; dimethindene and pheniramine; and piperidines, such as cyproheptadine and azatadine. These active agents, as well as additional antihistamines can also be incorporated in the composition of the present invention.

The composition of the present invention may also comprise an anti-inflammatory or antiallergic agent, wherein said agent reduces the occurrence of pro-inflammatory cytokines or inhibits the effect of pro-inflammatory cytokines.

Immunosuppressant agents, immunoregulating agents and immunomodulators are chemically or biologically derived agents that modify the immune response or the functioning of the immune system (as by the stimulation of antibody formation or the inhibition of white blood cell activity). Immunosuppressant agents and immunomodulators include, among other options, cyclic peptides, such as cyclosporine, tacrolimus, tresperimus, pimecrolimus, sirolimus (rapamycin), verolimus, laflunimus, laquinimod and imiquimod.

Hormones include steroid and non-steroid hormones. Steroid hormones can be selected from the group consisting of an androgen, an estrogen and a progestogen.

Exemplary androgens include testosterone, testosterone cipionate, testosterone decanoate, testosterone enantate, testosterone isocaproate, testosterone phenylpropionate, testosterone propionate, testosterone undecylate, 5α-dihydrotestosterone, dehydroepiandrosterone (also termed prasterone and DHEA), androstenedione, androstanediol, androsterone, androstenolone, prasterone enantate, prasterone sodium sulfate, ormeloxifene, mesterolone, fluoxymesterone, methyltestosterone, gestrinone, delmadinone, delmadinone acetate, chlormadinone, chlormadinone acetate, danazol and testolactone. Exemplary estrogens include estradiol, estradiol benzoate, estradiol cipionate, estradiol dipropionate, estradiol enantate, estradiol hexahydrobenzoate, estradiol phenylpropionate, estradiol valerate, polyestradiol phosphate, estriol, estriol sodium succinate, estriol succinate, polyestriol phosphate, quinestradol, ethinylestradiol, estrapronicate, mestranol, estrapronicate and equilin. Exemplary progestogens include progesterone, norethisterone, norethisterone acetate, norethisterone enantate, medroxyprogesterone acetate, delmadinone acetate, flugestone acetate, dydrogesterone, desogestrel, norgestrel, levonorgestrel, dydrogesterone, gestodene, chlormadinone acetate, dienogest, drospirenone, lynestrenol, tybolone, cyproterone acetate, megestrol acetate, nomegestrol acetate;

The term “keratolytically active agent” refers herein to a compound which loosens and removes the stratum corneum of the skin, or alters the structure of the keratin layers of skin.

Suitable keratolytically active agents include phenol and substituted phenolic compounds. Such compounds are known to dissolve and loosen the intracellular matrix of the hyperkeratinized tissue. Dihydroxy benzene and derivatives thereof have been recognized as potent keratolytic agents. Resorcinol (m-dihydroxybenzene) and derivatives thereof are used in anti-acne preparations. Hydroquinone (p-dihydroxybenzene), besides its anti-pigmentation properties, is also keratolytic.

Vitamin A and its derivatives, such as retinoic acid, isoretinoic acid, retinol and retinal are another preferred class of keratolytically active agents.

Another group of keratolytically active agents include alpha-hydroxy acids, such as lactic acid, glycolic acid, citric acid and malic acid and their respective salts and derivatives; and beta-hydroxy acids, such as Salicylic acid (o-hydroxybenzoic acid) and its salts and pharmaceutically acceptable derivatives, which typically possess anti-inflammatory, as well as keratolytic, activity. Yet, another class of preferred keratolytically active agents includes urea and its derivatives.

In one or more embodiments, the active agent is a retinoid. Retinoids include, for example, retinol, retinal, all trans retinoic acid and derivatives, isomers and analogs thereof. Etretinate, actiretin, isotretinoin, adapalene and tazarotene are further examples of said retinoid isomers and analogs.

In one or more embodiments, the active agent is a dicarboxylic acid. Exemplary pharmaceutically and cosmetically active dicarboxylice acids include azelaic acid, sebacic acid, adipic acid, fumaric acid and salts, isomers and analogs thereof.

In one or more embodiments, the active agent is a topical anesthetic. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, benzyl alcohol and phenol. Mixtures of such anesthetic agents may be synergistically beneficial.

In one or more embodiments, the active agent is an insecticide or an insect repellent agent. By way of example, insecticide can be selected selected from the group consisting of permethrin, hexachlorobenzene, carbamate, naturally occuring pyrethroids, permethrin, allethrin, malathion, piperonyl butoxide and any combination thereof at a therapeutically effective concentration. There are several types of insect repellents to use when protecting people and animals from flying or biting insects, spiders, ticks and mites. By way of example, these may include DEET (N,N-diethyl-m-toluamide), dimethyl phthalate, piperonyl butoxide and permethrin. The application of insecticides or insect repellent agents in foam is very convenient. Foam spreads easily, even over large and/or hairy areas. The film forming agent present in the foam composition helps retain the insecticide or insect repellent on the treated area for an extended period of time.

In one or more embodiments the active agent is a sunscreen agent. As used herein, sunscreen agents include both chemical and physical sunblocks. Suitable sunscreen agents may be organic or inorganic.

A wide variety of conventional organic sunscreen actives are suitable for use herein. Specific suitable sunscreen actives include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-propyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol)(6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-dibenzoylmethane.

A safe and effective amount of the organic sunscreen active is used, typically from about 1% to about 20%, more typically from about 2% to about 10% of the composition. Exact amounts will vary depending upon the sunscreen or sunscreens chosen and the desired Sun Protection Factor (SPF).

Inorganic sunscreens useful herein include the following metallic oxides; titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20%, preferably from about 0.5% to about 10%, more preferably from about 1% to about 5%, of the composition.

In one or more embodiments, the active agent is an anti cancer agent, i.e., an agent that has an effect in preventing, treating or alleviating the symptoms of cancer. Preferred anti cancer agents are agents that are capable of treating skin malignant tumors, such as basal cell carcinoma, squamous sell carcinoma, melanoma and Kaposi's sarcoma, as well as pre-cancerous conditions, such as actinic keratosis. In certain cases, topical cytotoxic and antiproliferative drugs are used to prevent, treat or alleviate the symptoms of such cancers.

In one or more embodiments, the active agent is a photodynamic therapy (PDT) agent. By way of example, such PDT agents can be selected from the group comprising modified porphyrins, chlorins, bacteriochlorins, phthalocyanines, naphthalocyanines, pheophorbides, purpurins, m-THPC, mono-L-aspartyl chlorin e6, bacteriochlorins, phthalocyanines, benzoporphyrin derivatives, as well as photosensitizer precursors, such as aminolevulinic acid (ALA).

In one or more embodiments, the active agent is an agent, useful in the treatment of burns, wounds, cuts and ulcers. The foam compositions of the present invention may comprise a combination of anti-infective agents (against bacteria, fungi and/or viruses), anti-inflammatory agents (steroidal and/or NSAIDs) and pain relieving components. Upon application, the foam spreads easily, covering the surface of the affected area, and without causing pain, and retains the activew agent(s) at the surface for an extended period of time, due to the beneficial effect of the film forming agent.

The foam compositions of the present invention, with or without further active ingredients, are suitable for the further application as “cosmeceutical” preparation (cosmetic products with therapeutic benefit), to treat “cosmetic” skin disorders, such as aging skin, wrinkles, hyperpigmentation (melasma, chloasma, freckles, etc.), scaly skin and other skin undesirable properties.

The term “cosmetic active agent” means the principle component or components that act to perform the primary function or functions of the cosmetic composition. Any cosmetic active agent is considered an “active agent” in the context of the present invention. The CTFA Cosmetic Ingredient Handbook describes a wide variety of non-limiting cosmetic active agents commonly used in the skin care industry, which are suitable for use in the compositions of the present invention. Examples of these ingredient classes include: abrasives, absorbents, aesthetic components such as fragrances, pigments, colorings/colorants, essential oils, astringents, clove oil, menthol, camphor, eucalyptus oil, eugenol, menthyl lactate, witch hazel distillate, anti-acne agents, anti-microbial agents (e.g., iodopropyl butylcarbamate), antioxidants, biological additives, cosmetic astringents, cosmetic biocides, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), and vitamins and derivatives thereof.

In one or more embodiments, the active agent is an agent, useful in the treatment of acne, wrinkles and scars. Examples of useful anti-acne actives include resorcinol, sulfur, salicylic acid and salicylates, alpha-hydroxy acids, nonsteroidal anti-inflammatory agents, benzoyl peroxide, retinoic acid, isoretinoic acid and other retinoid compounds, adapalene, tazarotene, azelaic acid and azelaic acid derivatives, antibiotic agents, such as erythromycin and clyndamycin, zinc salts and complexes, and combinations thereof, in a therapeutically effective concentration. Exemplary anti-wrinkle/anti-atrophy active agents suitable for use in the compositions of the present invention include sulfur-containing D and L amino acids and their derivatives and salts, particularly the N-acetyl derivatives; thiols; hydroxy acids (e.g., alpha-hydroxy acids such as lactic acid and glycolic acid and their derivatives and salts; or beta-hydroxy acids such as salicylic acid and salicylic acid salts and derivatives), urea, hyaluronic acid, phytic acid, lipoic acid; lysophosphatidic acid, skin peel agents (e.g., phenol, resorcinol and the like), vitamin B3 compounds (e.g., niacinamide, nicotinic acid and nicotinic acid salts and esters, including non-vasodilating esters of nicotinic acid (such as tocopheryl nicotinate), nicotinyl amino acids, nicotinyl alcohol esters of carboxylic acids, nicotinic acid N-oxide and niacinamide N-oxide), vitamin B5 and retinoids (e.g., retinol, retinal, retinoic acid, retinyl acetate, retinyl palmitate, retinyl ascorbate). In the case of dry, scaly skin (xerosis) and ichthyosis such agents can alleviate the symptoms by temporary relief of itching associated with these conditions.

In one or more embodiments, the active agent is an anti-oxidants or a radical scavenger. Anti-oxidants/radical scavengers such as ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid, gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts may be used.

In one or more embodiments, the active agent is a self-tanning active agent, such as dihydroxyacetone.

According to another embodiment, the active agent comprises solid matter or particulate matter i.e., material that is not soluble in the liquid carrier composition of the foamable composition. For definition purposes, solid matter shall mean material that is not soluble in the foamable composition more than 10% of the concentration intended to be included in said foamable composition. By way of example, the following classes of solid matter substances are presented: metallic oxides, such as titanium dioxide, zinc oxide, zirconium oxide, iron oxide; silicon containing materials such as silicone oxide and talc; carbon, for example in the form of amorphous carbon or graphite; insoluble oxidizing agents, such as benzoyl peroxide, calcium and magnesium hypochlorite; metallic Silver; cosmetic scrub materials, including, for example meals of strawberry seeds, raspberry seeds, apricot seeds, sweet almond, cranberry seeds; pigments.

It is further pointed out that unsaturated polyunsaturated fatty acids and polyunsaturated fatty acids, containing omega-3 and omega-6 fatty acids (e.g., linoleic and linolenic acid, gamma-linoleic acid (GLA), eicosapentaenoic acid (EPA) and docosahexaenoic acid (DHA), as well as their respective glyceride esters (also termed herein “unsaturated oils” and “poly-unsaturated oils”, respectively), are beneficial in the treatment of psoriasis and other skin inflammation conditions. Likewise, emollients and silicone oils exert moisture-retaining and skin protective effects on the skin. Thus in certain embodiments, a skin protective foam is provided, wherein the hydrophobic carrier comprises in full or in part, an organic liquid, selected from the group consisting of emollients, silicone oil and oils, rich in unsaturated oils and poly-unsaturated oils.

In one or more embodiments, a pharmaceutically or cosmetically active agent is an organic substance which is semi-liquid or liquid at ambient temperature. As such, a semi-liquid or liquid pharmaceutically or cosmetically active agent can be concurrently considered as an “active agent” and as an “organic carrier”, as defined herein. Non-limiting examples of semi-liquid or liquid pharmaceutically or cosmetically active agent include, but are not limited to permethrin, diethyl toluamide (DEET), dimethyl phthalate, and vitamin E (tocoferol). The semi-liquid or liquid pharmaceutically or cosmetically active agent can be included in the foamable composition as a sole organic carrier, or in combination with additional organic carriers, as detailed above.

According to certain embodiment, the active agent is selected from the group consisting of a solvent, a surface active agent, a foam adjuvant and a gelling agent, which are, on a case by case basis known to possess a therapeutic benefit.

Combinations of more than one active agent are contemplated. Thus, in certain embodiments, the composition contains at least two active agents. In further embodiments each of the at least two active agents exerts itsr therapeutic effect through at different mode of action, thus providing synergy and consequent synergistic therapeutic results.

In one or more embodiments, the active agent is encapsulated in particles, microparticles, nanoparticles, microcapsules, spheres, microspheres, nanocapsules, nanospheres, liposomes, niosomes, polymer matrix, nanocrystals or microsponges.

Optional Formulation Excipients

The composition of the present invention may further optionally include a variety of formulation excipients, which are added in order to fine-tune the consistency of the formulation, protect the formulation components from degradation and oxidation and modify their consistency. Such excipients may be selected, for example, from stabilizing agents, antioxidants, humectants, preservatives, colorant and odorant agents and other formulation components, used in the art of formulation.

“Alcohol Free”

Unlike the composition disclosed in WO 2004/071479, which contains a high concentration of a monohydric aliphatic alcohol, the composition of the present invention does not contain such amount alcohols. For the purpose of the present application, the term “alcohol free” shall mean that the composition contains no more than an incidental amount of an aliphatic alcohol, e.g. less than about 7.5% of any aliphatic alcohol, having one to six carbon atoms in their carbon backbone, or no more than 7.5% of any mixture of such aliphatic alcohols. Alcohols at these low levels are not considered to have a negative effect on skin or mucous membranes. In one or more embodiments, the foamable compositions do not contain any alcohol.

Propellants

Examples of suitable propellants include volatile hydrocarbons such as butane, propane, isobutane and fluorocarbon gases, or mixtures thereof.

In certain embodiments, fluorohydrocarbon propellants, other than chloro-fluoro carbons (CMCs) which are non-ozone-depleting propellants, are particularly useful in the production of a non-flammable foamable composition.

Such propellants include, but are not limited to hydrofluorocarbon (HFC) propellants, which contain no chlorine atoms, and as such, falls completely outside concerns about stratospheric ozone destruction by chlorofluorocarbons or other chlorinated hydrocarbons. Exemplary non-flammable propellants according to this aspect of the invention include propellants made by DuPont under the registered trademark Dymel, such as 1,1,1,2 tetrafluorethane (Dymel 134), and 1,1,1,2,3,3,3 heptafluoropropane (Dymel 227), 1,1,difluoro ethane (Dymel 152) and 1,1,1,3,3,3 hexafluoropropane. HFCs possess Ozone Depletion Potential of 0.00 and thus, they are allowed for use as propellant in aerosol products.

The propellant makes up about 5-25 wt % of the foamable composition. Aerosol propellants are used to generate and administer the foamable composition as a foam. The total composition including propellant, foamable compositions and optional ingredients is referred to as the foamable composition.

Composition and Foam Physical Characteristics and Advantages

A pharmaceutical or cosmetic composition manufactured using the foamable carrier of the present invention is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed.

The foamable composition of the present invention is stable, having an acceptable shelf-life of an year, or at least two years at ambient temperature, as revealed in accelerated stability tests. Organic carriers and propellants tend to impair the stability of emulsions and to interfere with the formation of a stable foam upon release from a pressurized container. It has been observed, however, that the foamable compositions according to the present invention are surprisingly stable. Following accelerated stability studies, they demonstrate desirable texture; they form fine bubble structures that do not break immediately upon contact with a surface, spread easily on the treated area and absorb quickly.

The composition should also be free flowing, to allow it to flow through the aperture of the container, e.g., and aerosol container, and create an acceptable foam.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

As further aspect of the foam is breakability. The breakable foam is thermally stable, yet breaks under sheer force. Sheer-force breakability of the foam is clearly advantageous over thermally induced breakability. Thermally sensitive foams immediately collapse upon exposure to skin temperature and, therefore, cannot be applied on the hand and afterwards delivered to the afflicted area.

The foam of the present invention has several advantages, when compared with hydroalcoholic foam compositions, such as described in WO 2004/071479:
(1) Breakability. The foam of the present invention is thermally stable. Unlike hydroalcoholic foam compositions of the prior art, the foam of the present invention is not “quick breaking”, i.e., it does not readily collapse upon exposure to body temperature environment. Sheer-force breakability of the foam is clearly advantageous over thermally induced breakability, since it allows comfortable application and well directed administration to the target area.
(2) Skin drying and skin barrier function. short chain alcohols are known to dry the skin and impair the integrity of the skin barrier. By contrast, including a film forming agent in the composition of the present invention foes not cause unwanted skin barrier damage.
(3) Irritability. Due to the lack of alcohol and improvement in skin barrier function, skin irritability is eliminated.

In terms of usability, the foamable composition is most advantageous, as revealed by clinical trials:
(i) Ease of application.
   When foam is released, it expands and allows easy spreading on the target area. This advantage is particularly meaningful in regards to the treatment of large skin surfaces.
   Upon application, the foam readily spreads and absorbs into the skin.
(ii) The Foam is Drip-Free
   The foam is not liquid and therefore does not leak when applied.
   This allows precise application, without the product being spread on clothes or other parts of the body.

Another property of the foam is specific gravity, as measured upon release from the aerosol can. Typically, foams have specific gravity of less than 0.12 g/mL; or less than 0.12 g/mL; or less than 0.08 g/mL, depending on their composition and on the propellant concentration.

Fields of Applications

According to one or more embodiments of the present invention, the foamable carrier and the foamable pharmaceutical or cosmetic composition of the present invention is intended for administration to an animal or a human subject. In one or more embodiments, the composition is intended to treat the skin, a body surface, a body cavity or a mucosal surface, e.g., the mucosa of the nose, mouth, eye, ear, respiratory system, vagina or rectum.

By including an appropriate active agent in the compositions of the present invention, the composition are useful in treating a patient having any one of a variety of dermatological disorders, which include inflammation as one or their etiological factors (also termed “dermatoses”), such as classified in a non-limiting exemplary manner according to the following groups:

Dermatitis including contact dermatitis, atopic dermatitis, seborrheic dermatitis, nummular dermatitis, chronic dermatitis of the hands and feet, generalized exfoliative dermatitis, stasis dermatitis; lichen simplex chronicus; diaper rash;

Bacterial infections including cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, erythrasma;

Fungal Infections including dermatophyte infections, yeast Infections; parasitic Infections including scabies, pediculosis, creeping eruption;

Viral Infections;

Disorders of hair follicles and sebaceous glands including acne, rosacea, perioral dermatitis, hypertrichosis (hirsutism), alopecia, including male pattern baldness, alopecia areata, alopecia universalis and alopecia totalis; pseudofolliculitis barbae, keratinous cyst;

Scaling papular diseases including psoriasis, pityriasis rosea, lichen planus, pityriasis rubra pilaris;

Benign tumors including moles, dysplastic nevi, skin tags, lipomas, angiomas, pyogenic granuloma, seborrheic keratoses, dermatofibroma, keratoacanthoma, keloid;

Malignant tumors including basal cell carcinoma, squamous cell carcinoma, malignant melanoma, Paget's disease of the nipples, Kaposi's sarcoma;

Reactions to sunlight, including sunburn, chronic effects of sunlight, photosensitivity;

Bullous diseases including pemphigus, bullous pemphigoid, dermatitis herpetiformis, linear immunoglobulin A disease;

Pigmentation disorders including hypopigmentation such as vitiligo, albinism and postinflammatory hypopigmentation and hyperpigmentation such as melasma (chloasma), drug-induced hyperpigmentation, postinflammatory hyperpigmentation;

Disorders of cornification including ichthyosis, keratosis pilaris, calluses and corns, actinic keratosis;

Pressure sores, open wounds, chronic wounds, open ulcers and burns;

Disorders of sweating; and

Inflammatory reactions including drug eruptions, toxic epidermal necrolysis; erythema multiforme, erythema nodosum, granuloma annulare.

Upon application of the foam, incorporating an active agent, the foam spreads easily, covering the surface of the affected area, and without causing pain. It retains the active agent(s) at the surface for an extended period of time, due to the beneficial effect of the film forming agent.

The same advantage is expected when the composition is topically applied to a body cavity or mucosal surfaces, including, but not limited to the cranial cavity, the thoracic cavity, the abdominal cavity, the ventral cavity, the vagina, the rectum and penile cavities, the urinary tract, the nasal cavity, the mouth, the eye, the ear the peritoneum, the large and small bowel, the caecum, bladder, and stomach, the cavity between the uterus and the fallopian tubes, the ovaries and other body areas, which may accept topically-applied products. The composition of the present invention is suitable to treat conditions of a body cavity and a mucosal membrane, such as post-surgical adhesions, Chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

According to one or more embodiments of the present invention, the compositions are also useful in the therapy of non-dermatological disorders by providing transdermal or transmucosal delivery of an active agent that is effective against non-dermatological disorders.

In one or more embodiments, the disorder is a health abnormality that responds to treatment with a hormone. A typical example of such abnormality is sexual dysfunction in men and women whereby androgen therapy is successfully used to restore sexual function. Other non-limiting examples of disorders/medical indications that are in the scope of treatment with a hormone according to the present invention are androgen deficiency, estrogen deficiency, growth disorders, hypogonadism, cancer, vasomotor symptoms, menopausal disorders, vulvar and vaginal atrophy, urethritis, hypoestrogenism, osteoarthritis, osteoporosis, uterine bleeding, Hirsutism, Virilization, ovarian tumors, hypothalamic pituitary unit diseases, testicular tumors, prostate cancer, hypopituitarism, Klinefelter's syndrome, testicular feminisation, orchitectomy, vasomotor symptoms (such as “hot flashes”) associated with the menopause, metabolic abnormalities and mood disturbances.

The following examples exemplify the therapeutic kits and pharmacological compositions and methods described herein. The examples are for the purposes of illustration only and are not intended to be limiting of the invention.

The following examples exemplify the therapeutic compositions and pharmacological compositions and methods described herein. The examples are for the purposes of illustration only and are not intended to be limiting of the invention.

### EXAMPLE 1

### Insect Repellent Composition Containing a Film Forming Polymer

| Ingredient | % w/w |
|---|---|
| Diethyltoluamide | 25.00 |
| Glycerine | 5.00 |
| PEG 400 | 5.00 |
| Stearic acid | 4.00 |
| Span 60 | 4.00 |
| Tween 60 | 2.00 |
| Dermacryl 79 (film forming agent) | 0.7 |
| Isosearic Acid | 0.50 |
| Triethanolamine | 0.46 |
| Phenonip (preservative) | 0.25 |
| Pemulen TR2 (film forming agent) | 0.06 |
| Propellant (propane and butane) | 6.00 |
| Water | 47.03 |
| | 100.00 |

### EXAMPLE 2

### Insect Repellency Test in Humans

The efficacy of the foamable composition of Example 1 as repellent against Aedes aegypti mosquitoes was tested under laboratory conditions, in comparison with the same composition without the film forming polymer Dermacryl and Pemulen.

The mosquitoes used in this work were laboratory-reared, sugar-fed, 3- to 5-days-old adult Aedes aegypti. Before the test, the mosquitoes were starved for 24 h. The test was carried out from 8.30 to 16.30.

The test was conducted in a room maintained at 23±2° C. Human volunteers were used, whereby one of the repellants was applied to one bare forearm and hand and another formulation to the other forearm and hand. Then, every hour for during the test period, each volunteer put each treated arm into a mosquito cage, containing about 150-200 starved Aedes aegypti adult mosquitoes for 10 minutes. The number of mosquitoes landing on each of the arms was counted during each 10-minute exposure (8 replicas) for a total period of 8-hours. The number of bites for each 10-minute exposure period was also recorded.

For each of the volunteers, the number of landed mosquitoes and bites on the bare untreated forearm of eight human volunteers during 10 min in mosquito (Aedes aegypti) cages each hour for 8 hours is presented in the following table:

| | DEET Foam without Dermacryl | | | | | | DEET Foam with Dermacryl | | | | | |
|---|---|---|---|---|---|---|---|---|---|---|---|---|
| | Volunteer # 1 | | | Volunteer # 2 | | | Volunteer # 1 | | | Volunteer # 2 | | |
| | Number | | No. | Number | | No. | Number | | No. | Number | | No. |
| Exposure | of landed | | of | of landed | | of | of landed | | of | of landed | | of |
| (Hours) | mosquitoes | Mean | bites | mosquitoes | Mean | bites | mosquitoes | Mean | bites | mosquitoes | Mean | bites |
| 0 | 0 0 0 1 | 0.25 | 0 | 0 0 0 0 | 0.25 | 0 | 0 0 0 0 | 0.37 | 0 | 0 0 1 2 | 0.5 | 0 |
| | 0 1 0 0 | | | 0 0 1 1 | | | 0 1 1 1 | | | 1 0 0 0 | | |
| 1 | 1 0 0 0 | 0.12 | 0 | 0 0 0 0 | 0 | 0 | 0 0 1 0 | 0.12 | 0 | 0 0 0 0 | 0 | 0 |
| | 0 0 0 0 | | | 0 0 0 0 | | | 0 0 0 0 | | | 0 0 0 0 | | |
| 2 | 0 0 0 1 | 0.12 | 0 | 0 0 0 0 | 0.12 | 0 | 0 0 0 0 | 0 | 0 | 0 0 0 0 | 0 | 0 |
| | 0 0 0 0 | | | 0 0 1 0 | | | 0 0 0 0 | | | 0 0 0 0 | | |
| 3 | 2 0 0 0 | 0.37 | 0 | 0 0 0 0 | 0.12 | 0 | 0 0 0 0 | 0.37 | 0 | 0 0 0 0 | 0 | 0 |
| | 0 0 0 1 | | | 0 0 1 0 | | | 0 2 1 0 | | | 0 0 0 0 | | |
| 4 | 1 1 0 1 | 0.75 | 0 | 0 0 0 0 | 0 | 0 | 1 1 0 1 | 0.5 | 0 | 0 1 1 0 | 0.25 | 0 |
| | 0 1 1 1 | | | 0 0 0 0 | | | 1 0 0 0 | | | 0 0 0 0 | | |
| 5 | 3 2 0 0 | 1 | 0 | 0 0 1 0 | 0.25 | 0 | 2 1 1 0 | 0.75 | 0 | 0 0 0 0 | 0 | 0 |
| | 1 1 0 1 | | | 1 0 0 0 | | | 0 0 1 1 | | | 0 0 0 0 | | |
| 6 | 2 1 1 2 | 1 | 0 | 0 0 0 0 | 0.37 | 0 | 2 2 2 3 | 1.37 | 0 | 0 1 0 0 | 0.12 | 0 |
| | 1 0 0 1 | | | 1 1 1 0 | | | 1 1 0 0 | | | 0 0 0 0 | | |
| 7 | 3 3 3 3 | 3 | 6 | 1 0 0 0 | 0.25 | 0 | 1 0 1 2 | 1.37 | 1 | 1 0 0 0 | 0.12 | 0 |
| | 3 2 3 4 | | | 1 0 0 0 | | | 3 2 1 1 | | | 0 0 0 0 | | |
| 8 | 3 5 5 4 | 4.12 | 17 | 0 0 0 1 | 0.62 | 0 | 3 3 3 0 | 1.62 | 3 | 0 0 0 1 | 0.37 | 0 |
| | 4 5 4 3 | | | 0 0 2 2 | | | 0 2 2 0 | | | 0 0 0 2 | | |

As seen in the above table, the DEET Foam with Dermacryl was superior to the same composition with no Dermacryl. It was protective for a longer period of time and the total number of bites was far less when the DEET Foam with Dermacryl was used.

### EXAMPLE 3

### Foamable Composition Containing Aculyn and Pemulen for Treating Atopic Dermatitis

| Ingredient | % w/w |
|---|---|
| Mineral Oil | 3.00 |
| Shea Butter | 2.00 |
| Avocado Oil | 4.00 |
| C12-C15 Alkyl Benzoate | 4.00 |
| Cyclomethicone (Dow Corning 245) | 0.50 |
| Stearyl Dimethicone (Dow Corning 2502) | 2.00 |
| Stearic Acid | 0.80 |
| Polyoxyl 2 Stearyl Ether (Brij72) | 0.75 |
| Polyoxyethylene 21 Stearyl Ether (Brij721) | 1.50 |
| Behenyl Alcohol | 0.40 |
| Acrylates/C10-30 Alkylacrilate Crosspolymer | 0.10 |
| (Pemulen TR2, film forming agent) | |
| Bis PEG/PPG-18Methyl Ether Dimethyl Silane | 1.00 |
| (Dow Corning 2501, film forming agent) | |
| Propylene Glycol | 3.00 |
| Glycerin | 5.00 |
| PEG150/Stearyl Alcohol/SMDI Copolymer (Aculyn46) | 1.00 |
| Aloe Vera extract | 0.30 |
| Triethanolamine (TEA) | 0.15 |
| Water | 60.10 |
| Disodium EDTA | 0.10 |
| Sodium Hyaluronate (1%) | 0.50 |
| Licorice Extract | 0.50 |
| Alpha Bisabolol | 0.30 |
| Tocopheryl Acetate | 0.50 |
| Allantoin | 0.50 |
| Propane/Butane/Isobutane | 8.00 |
| Total | 100.00 |

The composition of Example 3 contains two film forming agents and a series of active agents that are known to affect skin inflammation, including avocado oil, cyclomethicone, dimethicone, aloe vera extract, sodium hyaluronate, licorice extract, alpha bisabolol, tocopheryl acetate and allantoin. Conceivably, at least two of these active agents are required to attain effective treatment.

### EXAMPLE 4

### A Human Study, to Test the Skin Hydration Effect of a Foamble Composition Containing Aculyn and Pemulene for Treating Atopic Dermatitis

Several skin disorders are characterized, among other etiological factors, by severe skin dryness, as exemplified by psoriasis, atopic dermatitis, xerosis and ichthyosis. Atopic dermatitis is a chronic disease that affects the skin. In atopic dermatitis, the skin becomes extremely itchy. Scratching leads to redness, swelling, cracking, excreting clear fluid, and finally, crusting and scaling. In most cases, there are periods when the disease is worse (called exacerbations or flares) followed by periods when the skin improves or clears up entirely (called remissions). As some children with atopic dermatitis grow older, their skin disease improves or disappears altogether, although their skin often remains dry and easily irritated. In others, atopic dermatitis continues to be a significant problem in adulthood.

It is commonly accepted that emollients are a standard of care, steroid sparing and useful for both prevention and maintenance therapy as stated in a recent Guidelines of Care for Atopic Dermatitis by the American Academy of Dermatology.

The objective of the present study was to assess the skin hydration properties of the composition of Example 3, in comparison with the commercially available medicated cream, namely Atopiclair that does not contain film-forming agents.

Six human volunteers were treated once with the composition of Example 3 on one forearm and with Atopiclair on the other forearm. A untreated site remained as control site. Skin hydration values were determined at each designated treatment site, using the Corneometer CM825 instrument (Courage+Khazaka, Koln, Germany) at constant room conditions (24° C. and 40% humidity).

The following table present skin hydration values, as measured by Corneometer for the composition of Example 3, Atopiclair and no-treatment, at Baseline and 1, 3 and 6 hours after treatment, as well as the percent change of skin hydration from Baseline and p Values (vs. Baseline and within treatments) during the study.

As shown in the table, non-treated areas maintained practically constant hydration values throughout the study. The composition of Example 3 afforded elevated skin hydration of about 20-45% during the 6-hours follow up, while Atopiclair was effective for one hour only. Notable, the change from baseline was statistically significant for both the composition of Example 3 throughout the 6-hours assessment (T-Test). Moreover, the superiority of the composition of Example 3 to Atopiclair was statistically evident at the 3 hours and 6 hours measurements.

TABLE
Skin hydration values for the composition of Example 3, Atopiclair
and no-treatment, at Baseline and 1, 3 and 6 hours after treatment,
the percent change of skin hydration from Baseline and p Values
(vs. Baseline and within treatments)

| | | No Treatment | Example 3 | Atopiclair |
|---|---|---|---|---|
| Base- | Skin hydration value | 43.75 | 45.16 | 46.50 |
| line | STD | 4.19 | 4.48 | 4.36 |
| 1 | Skin hydration value | 45.50 | 55.62 | 52.04 |
| Hour | STD | 4.25 | 8.77 | 6.05 |
| | % Change from baseline | 3% | 23% | 12% |
| | p Value (vs. Baseline) | NA | 0.0311 | 0.3092 |
| | P Value (vs. Atopiclair) | | 0.5213 | |
| 3 | Skin hydration value | 45.62 | 62.50 | 47.75 |
| Hour | STD | 5.32 | 7.47 | 5.11 |
| | % Change from baseline | 4% | 38% | 3% |
| | p Value (vs. Baseline) | NA | 0.0046 | 0.3945 |
| | P Value (vs. Atopiclair) | | 0.0033 | |
| 6 | Skin hydration value | 48.58 | 65.04 | 49.50 |
| Hour | STD | 4.41 | 8.99 | 5.12 |
| | % Change from baseline | 11% | 44% | 6% |
| | p Value (vs. Baseline) | NA | 0.001 | 0.700 |
| | P Value (vs. Atopiclair) | | 0.001 | |

### EXAMPLE 5

### Foamable Composition Containing Aculyn and Pemulen as Film Forming Agents

| Ingredient | % w/w |
|---|---|
| Mineral Oil | 1.50 |
| Dow Corning 245 (film forming agent) | 0.70 |
| Dow Corning 2502 (film forming agent) | 1.00 |
| Alkyl Benzoate | 8.00 |
| Stearic Acid | 1.00 |
| Brij 72 | 1.00 |
| Brij 721 | 2.00 |
| Propylen glycol | 5.00 |
| Stearyl Alcohol | 1.00 |
| Pemulen TR2 (film forming agent) | 0.05 |
| Glycerin | 3.00 |
| Dow 2501 | 1.50 |
| Aculyn 46 (film forming agent) | 1.80 |
| TEA | 0.10 |
| Water | 63.90 |
| Phenonip | 0.25 |
| Fragance | 0.20 |
| Propellant | 8.00 |
| Total | 100.00 |

### EXAMPLE 6

### Foamable Composition Containing Chitosan as Film Forming Agent

| Ingredient | % w/w | % w/w | % w/w |
|---|---|---|---|
| Zinc oxide | 10.00 | 10.00 | 10.00 |
| Mineral oil | 20.00 | 20.00 | 20.00 |
| IPP | 10.00 | 10.00 | 10.00 |
| Beeswax | 2.00 | 2.00 | 2.00 |
| Glyceryl oleate | 1.00 | 1.00 | 1.00 |
| Lanolin | 5.00 | 5.00 | 5.00 |
| Lipocol C 2 | 3.00 | 3.00 | 3.00 |
| Sucrose ester SP10 | 1.00 | 1.00 | 1.00 |
| Aloe vera extract | 0.10 | 0.10 | 0.10 |
| Alpha Bisabolol | 0.20 | 0.20 | 0.20 |
| Xanthan gum | — | 0.40 | 0.40 |
| Chitosan (film forming agent)* | 0.18 | 0.18 | 0.18 |
| Lactic acid | 0.35 | 0.35 | 0.35 |
| D-Panthenol | 4.00 | 4.00 | 4.00 |
| Benzalkonium chlorid | 0.20 | 0.20 | 0.20 |
| Propellant | 8.00 | 8.00 | 8.00 |
| Water | To 100.00 | To 100.00 | To 100.00 |
| Foam Quality | G | G | E |
| Density | 0.1141 | 0.0897 | 0.1058 |

*CAS-no.: 9012-76-4; Formula: (C6H11O4N)ₙ; Mol. weight: 30.000-1.000.000 Dalton I

Example 6 provides a foam based on chitosan as a film forming agent. The composition contains zinc oxide, lactic acid, aloe vera extract, alpha bisabolol and panthenol as active agents.

### EXAMPLE 7

### Foamable Composition Containing Natrosol as Film Forming Agent

| | % w/w |
|---|---|
| Ingredient | |
| IPM/MCT | 6.00 |
| Glyceryl monooleate | 2.00 |
| Glyceryl monostearate | 1.00 |
| Span 60 | 1.00 |
| Arlamol E | 0.50 |
| Xanthan Gum | 0.30 |
| Hydroxyethylcellulose (Natrasol 250 HF; film forming agent) | 0.60 |
| Glycerin | 5.00 |
| Phenonip | 0.60 |
| Lactic acid or Sodium hydroxide | To pH = 5 |
| Propellant | 6.00 |
| Water | To 100 |
| Centrifugation | |
| 10000/3 min | stable |
| 10000/10 min | stable |
| Foam Quality | E |

What is claimed is:

1. A foamable composition, including:
(1) about 6% to about 70% by weight of at least one organic carrier;
(2) about 0.1% to about 5% by weight of at least one surface-active agent;
(3) about 0.01% to about 5% by weight of at least one film forming agent;
(4) water; and
(5) about 3% to about 25% by weight of the total composition of at least one liquefied or compressed gas propellant.

2. The composition of claim 1 wherein said composition is substantially alcohol-free.

3. The composition of claim 1, further including about 0.1% to about 5% by weight of a therapeutically active foam adjuvant is selected from the group consisting of a fatty alcohol having 15 or more carbons in their carbon chain, a fatty acid having 16 or more carbons in their carbon chain, fatty alcohols derived from beeswax and including a mixture of alcohols, a majority of which has at least 20 carbon atoms in their carbon chain, a fatty alcohol having an double bond, a fatty acid having an double bond, a branched fatty alcohol, a branched fatty acid, a fatty acid substituted with a hydroxyl group, cetyl alcohol; stearyl alcohol; arachidyl alcohol, behenyl alcohol, 1-triacontanol; hexadecanoic acid, stearic acid, arachidic acid, behenic acid, octacosanoic acid, 12-hydroxy stearic acid and mixtures thereof.

4. The composition of claim 1, further including about 0.01% to about 5% by weight of a polymeric additive selected from a gelling agent, a bioadhesive agent and a phase change agent.

5. The composition of claim 1, wherein the film forming agent is a water-insoluble alkyl cellulose or hydroxyalkyl cellulose.

6. The composition of claim 5, wherein the film forming agent is selected from the group consisting of alkyl cellulose, hydroxyalkyl cellulose, ethyl cellulose, propyl cellulose, butyl cellulose, cellulose acetate, hydroxypropyl cellulose, hydroxybutyl cellulose, and ethylhydroxyethyl cellulose

7. The composition of claim 1, wherein the film forming agent is a hydrophobic, high molecular weight carboxylated acrylic copolymer.

8. The composition of claim 7, wherein the film forming agent is selected from the group consisting of DERMACRYL-LT, DERMACRYL-79 and DERMACRYL-AQF.

9. The composition of claim 1, wherein the film forming agent is an anionic hydrophobically modified alkali-soluble acrylic polymer emulsion.

10. The composition of claim 9, wherein the film forming agent is selected from the group consisting of acrylates/steareth-20 methacrylate copolymer, acrylates/laureth-25 methacrylate copolymer, acrylates/beheneth-25 methacrylate copolymer, PRG-150/stearyl alcohol/SMDI and analogous acrylate copolymers.

11. The composition of claim 1, wherein the film forming agent is selected from the group consisting of hydrophobically-modified polysaccharides, hydrophobically-modified ethoxylated urethanes, Acrylates/C10-30 Alkyl Acrylate Cross-Polymer, Acrylates/C10-30 alkyl acrylate crosspolymer, cetyl hydroxyethyl cellulose, acrylates/vinyl isodecanoate, acrylates/steareth-20 itaconate copolymer, acrylates/ceteth-20 itaconate copolymer and acrylates/aminoacrylates/C10-30 alkyl PEG 20 itaconate copolymer, polyvinylpyrrolidone/vinylacetate, polyurethane-1, polyvinylcaprolactam, polyvinylpyrrolidone/polyvinylcaprolactam, polyvinylpyrrolidone/dimethylaminopropylmethacrylamide, polyvinylpyrrolidone/polyvinylcaprolactam/dimethylaminopropylmethacrylamide, isobutylene ethylmaleimide/hydroxyethylmaleimide, polyvinyl-pyrrolidone/dimethylaminoethylmethacrylate, quaternized polyvinyl-pyrrolidone/di-methylaminoethylmethacrylate, polyvinylpyrrolidone/polyvinylcaprolactam/dimethyl-aminoethylmethacrylate, poly(vinylacetate/crotonates/vinylneodecanoate and octylacrylamide/acrylate/butylaminoethylmethacrylate.

12. The composition of claim 1, wherein the film forming agent is chitosan.

13. The composition of claim 1, wherein the organic carrier is selected from the group consisting of a hydrophobic organic carrier, an emollient, a polar solvent, and mixtures thereof.

14. The composition of claim 13, wherein the organic carrier is selected from the group consisting of:
i. Mineral oils;
ii. liquid oils originating from vegetable, marine or animal sources;
iii. liquid oils, selected from the group consisting of a saturated oil, an unsaturated oil and a polyunsaturated oil;
iv. oils selected from the group consisting of olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil and evening primrose oil or mixtures thereof, in any proportion;
v. oils comprising at least one unsaturated fatty acid, selected from the group consisting of omega-3 fatty acids, omega-6 fatty acids, linoleic acid, linolenic acid, gamma-linoleic acid (GLA), eicosapentaenoic acid (EPA) and docosahexaenoic acid (DHA);
vi. Unsaturated oils that possesses therapeutically-beneficial properties;
vii. essential oils;
viii. plant-derived oils;
ix. silicone oils;
x. silicone oils selected from the group consisting of polyalkyl siloxane, polyaryl siloxane, polyalkylaryl siloxane and polyether siloxane copolymer, polydimethylsiloxane(dimethicone) and poly(dimethylsiloxane)-(diphenyl-siloxane) copolymers;
xi. emollients selected from the group consisting of hexyleneglycol, propylene glycol, isostearic acid derivatives, isopropyl palmitate, isopropyl isostearate, isopropyl myristate, ethyl acetate, butyl acetate, methyl propionate, capric/caprylic triglycerides, octylmyristate, dodecyl-myristate; diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids and octyl hydroxystearate;
xii. A polar solvent, selected from the group consisting of a polyol, such as glycerol (glycerin), propylene glycol, hexylene glycol, diethylene glycol, a propylene glycol n-alkanols, a terpene, a di-terpene, a tri-terpene, a terpen-ol¹, limonene, menthol, dioxolane, ethylene glycol, a glycol, a sulfoxide, dimethylsulfoxide, dimethylformanide, methyl dodecyl sulfoxide, dimethylacetamide, monooleate of ethoxylated glycerides, 1-dodecylazacycloheptan-2-one and 2-(n-nonyl)-1,3-dioxolane;
xiii. Polyethylene glycol;
xiv. A Polyethylene glycol selected from the group consisting of PEG200, PEG300, PEG400, PEG600, PEG 4000, PEG 6000 and PEG 10000;
xv. A pharmaceutically or cosmetically active agent, which is semi-liquid or liquid at ambient temperature; and
xvi. A pharmaceutically or cosmetically active agent selected from the group consisting of permethrin, diethyl toluamide, dimethyl phthalate and vitamin E.

15. The composition of claim 1, wherein the organic carrier comprises a hydrophobic carrier/emollient and a polar solvent in a ratio of between about 8:1 and 1:4.

16. The composition of claim 1, wherein the foamable composition is selected from the group consisting of an oil-in-water emulsion and a water-in-oil emulsion.

17. The composition of claim 1, wherein upon release from the container, a shear-sensitive foam, having a density range selected from (1) between about 0.02 gr/mL and about 0.1 gr/mL, and (2) between about 0.02 gr/mL and about 0.1 gr/mL, is produced.

18. The composition of claim 1, further comprising at least one active agent.

19. The composition of claim 18, wherein the active agent is selected from the group consisting of a drug, a cosmetic active agent, an anti-infective, an antibiotic, an antibacterial agent, a disinfectant, an antifungal agent, an antiviral agent, an antiparasitic agent, an anti-inflammatory agent, an anti-allergic agent, a steroid, a steroidal anti-inflammatory agent, a nonsteroidal anti-inflammatory drug, an immunosuppressive agent, an immunomodulator, an immunoregulating agent, a hormone, a keratolytically active agent, an alpha hydroxyl acid, a beta-hydroxy acid, vitamin A, a vitamin A derivative, a retinoid, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a burn healing agent, an anesthetic, a protein, a peptide, a neuropeptide, a allergen, an immunogenic substance, a haptene, an oxidizing agent, an antioxidant, a dicarboxylic acid, an antiproliferative agent, an anticancer agent, a photodynamic therapy agent, a sunscreen agent, an anti-wrinkle agent, a radical scavenger, a metal oxide, an anti wrinkle agent, an anti-acne agent, a skin whitening agent, a self tanning agent, an anti-cellulite agent, a skin protective agent, a masking agent, an anti-wart agent, a refatting agent, a lubricating agent and mixtures thereof at any proportion;

20. The composition of claim 18, wherein the active agent is selected from the group consisting of
i. anti-Infective agents selected from an antibiotic agent, an antibacterial agent, an anti-fungal agent, an anti-viral agent and an anti-parasite agent;
ii. agents suitable to kill gram positive and gram-negative bacteria, protozoa, aerobic bacteria and anaerobic bacteria;
iii. antibiotic agents selected from the classes consisting of beta-lactam antibiotics, synthetic and semi-synthetic penicillins, aminoglycosides, ansa-type antibiotics, anthraquinones, antibiotic azoles, antibiotic glycopeptides, macrolides, antibiotic nucleosides, antibiotic peptides, antibiotic polyenes, antibiotic polyethers, quinolones, fluoroquinolnes, antibiotic steroids, cyclosporines, sulfonamides, tetracycline, chloramphenicol, dicarboxylic acids, salicylates, antibiotic metals, oxidizing agents, substances that release free radicals and/or active oxygen, cationic antimicrobial agents, quaternary ammonium compounds, biguanides, triguanides, bisbiguanides and analogs and polymers thereof and naturally occurring antibiotic compounds;
iv. antibiotic agents selected from the classes consisting of a strong oxidant and a free radical liberating compounds;
v. antibiotic agents selected from the group consisting of iodine, chlorohexidine and benzoyl peroxide;
vi. antifungal agents selected from the group consisting of an azole, a diazole, a triazole, miconazole, ketoconazole, clotrimazole, econazole, mebendazole, bifonazole, butoconazole, fenticonazole, isoconazole, oxiconazole, sertaconazole, sulconazole, thiabendazole, tiaconazole, fluconazole, itraconazole, ravuconazole and posaconazole;
vii. antifungal agents selected from the group consisting of griseofulvin, ciclopirox, amorolfine, terbinafine, Amphotericin B, potassium iodide, flucytosine (5FC) and any combination thereof at a therapeutically effective concentration;
viii. antiviral agents selected from the group consisting of acyclovir, famciclovir, gancyclovir, valganciclovir and abacavir;
ix. corticosteroids selected from the group consisting of hydrocortisone, hydrocortisone acetate, desonide, Betamethasone, betamethasone valerate, betamethasone dipropionate, betamethasone-17-benzoate, clobetasone-17-butyrate, flucinonide, fluocinolone acetonide, alcometasone dipropionate, mometasone furoate, prednicarbate, triamcinolone acetonide, methylprednisolone aceponate, halcinonide, triamcinolone acetonide, halobetasol and clobetasol-17-propionate;
x. nonsteroidal anti-inflammatory agents selected from the group consisting of an oxicam, piroxicam, isoxicam, tenoxicam, sudoxicam, a salicylate, salicylic acid, ethyl salicylate, methyl salycilate, aspirin, disalcid, benorylate, trilisate, safapryn, solprin, diflunisal, and fendosal, an acetic acid derivative, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, a fenamate, mefenamic, meclofenamic, flufenamic, niflumic, tolfenamic acids; a propionic acid derivative, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofenic, a pyrazole, phenylbutazone, oxyphenbutazone, feprazone, azapropazone and trimethazone;
xi. compounds having the capacity to prevent, alleviate the symptoms of, treat or cure inflammation processes;
xii. antiallergic agents selected from the group consisting of a thylenediamine, pyrilamine, antazoline and methapyrilene, a tripelennamine phenothiazine, promethazine, methdilazine, trimeprazine, an ethanolamine, diphenhydramine, bromodiphenhydramine, carbinoxamine, clemastine, dimenhydrinate, diphenylpyraline, doxylamine, phenyltoxamine, a piperazine, cyclizine, buclizine, chlorcyclizine, hydroxyzine, meclizine, thiethylperazine, an alkylamine, brompheniramine, pyrrobutamin, desbrompheniramine, tripolidine, dexchlorpherniramine, chlorpheniramine; dimethindene, pheniramine, a piperidine, cyproheptadine and azatadine;
xiii. agents that reduce the occurrence of pro-inflammatory cytokines or inhibits the effect of pro-inflammatory cytokines;
xiv. immunoregulating agents selected from the group consisting of an immunoregulating cyclic peptide, cyclosporine, tacrolimus, tresperimus, pimecrolimus, sirolimus, verolimus, laflunimus, laquinimod and imiquimod;
xv. hormones selected from the group consisting of steroid and non-steroid hormones;
xvi. hormones selected from the group consisting of testosterone, testosterone cipionate, testosterone decanoate, testosterone enantate, testosterone isocaproate, testosterone phenylpropionate, testosterone propionate, testosterone undecylate, 5α-dihydrotestosterone, dehydroepiandrosterone, androstenedione, androstanediol, androsterone, androstenolone, prasterone enantate, prasterone sodium sulfate, ormeloxifene, mesterolone, fluoxymesterone, methyltestosterone, gestrinone, delmadinone, delmadinone acetate, chlormadinone, chlormadinone acetate, danazol, testolactone, estradiol, estradiol benzoate, estradiol cipionate, estradiol dipropionate, estradiol enantate, estradiol hexahydrobenzoate, estradiol phenylpropionate, estradiol valerate, polyestradiol phosphate, estriol, estriol sodium succinate, estriol succinate, polyestriol phosphate, quinestradol, ethinylestradiol, estrapronicate, mestranol, estrapronicate, equilin, progesterone, norethisterone, norethisterone acetate, norethisterone enantate, medroxyprogesterone acetate, delmadinone acetate, flugestone acetate, dydrogesterone, desogestrel, norgestrel, levonorgestrel, dydrogesterone, gestodene, chlormadinone acetate, dienogest, drospirenone, lynestrenol, tybolone, cyproterone acetate, megestrol acetate and nomegestrol acetate;
xvii. keratolytically active agents selected from the group consisting of phenol, a substituted phenolic compound, dihydroxy benzene, resorcinol, hydroquinone, lactic acid, glycolic acid, citric acid, malic acid, a beta-hydroxy acid, salicylic acid and urea;
xviii. retinoids selected from the group consisting of retinol, retinal, all trans retinoic acid, retinyl acetate, retinyl palmitate, retinyl ascorbate, etretinate, actiretin, isotretinoin, adapalene and tazarotene;
xix. dicarboxylic acids selected from the group consisting of azelaic acid, sebacic acid, adipic acid, fumaric acid;
xx. topical anesthetic agents selected from the group consisting of benzocaine, lidocaine, bupivacaine, chlorprocaine, dibucaine, etidocaine, mepivacaine, tetracaine, dyclonine, hexylcaine, procaine, cocaine, ketamine, pramoxine, benzyl alcohol and phenol;
xxi. insecticides or an insect repellent agents selected from the group consisting of permethrin, hexachlorobenzene, carbamate, a naturally occurring pyrethroid, permethrin, allethrin, malathion, piperonyl butoxide, diethyl-m-toluamide), dimethyl phthalate;
xxii. sunscreen agents selected from the group consisting of p-aminobenzoic acid, p-dimethylaminobenzoic acid, an anthranilate, an o-amino-benzoate ester a salicylate esters, a cinnamic acid derivative, a-phenyl cinnamonitrile, butyl cinnamoyl pyruvate, a dihydroxycinnamic acid derivative, umbelliferone, methylumbelliferone, methylaceto-umbelliferone, a trihydroxy-cinnamic acid derivative, esculetin, methylesculetin, daphnetin, esculin, daphnin, diphenylbutadiene, stilbene, dibenzalacetone, benzalacetophenone, a naphtholsulfonate, 2-naphthol-3,6-disulfonic, 2-naphthol-6,8-disulfonic acids, dihydroxynaphthoic acid, an o-hydroxybiphenyldisulfonate, a p-hydroxybiphenyldisulfonate, a coumarin derivative, a diazole, 2-acetyl-3-bromoindazole, phenyl benzoxazole, methyl naphthoxazole, an aryl benzothiazoles, a quinine salt, a quinoline derivative, 8-hydroxyquinoline, 2-phenylquinoline, a; hydroxy- or methoxy-substituted benzophenone, uric acid, violuric acids, tannic acid, hexaethylether, hydroquinone; a benzophenones, oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane, a metallic oxide, titanium dioxide, zinc oxide, zirconium oxide and iron oxide;
xxiii. photodynamic therapy agents selected from the group consisting of modified porphyrins, chlorins, bacteriochlorins, phthalocyanines, naphthalocyanines, pheophorbides, purpurins, m-THPC, mono-L-aspartyl chlorine, bacteriochlorins, phthalocyanines, benzoporphyrin derivatives and aminolevulinic acid;
xxiv. agents capable of treating a disorder selected from acne, wrinkles and scars.
xxv. active agents selected from the group consisting of a phenol, resorcinol, sulfur, salicylic acid, a sulfur-containing amino acid, N-acetyl derivatives, a thiol, hyaluronic acid, phytic acid, lipoic acid, lysophosphatidic acid, vitamin B, niacinamide, nicotinic acid, tocopheryl nicotinate, a nicotinyl amino acid, a nicotinyl alcohol ester of carboxylic acids, nicotinic acid N-oxide and niacinamide N-oxide;
xxvi. anti-oxidant or a radical scavengers selected from the group consisting of ascorbic acid, an ascorbyl ester of fatty acids, magnesium ascorbyl phosphate, sodium ascorbyl phosphate, ascorbyl sorbate, tocopherol, tocopherol sorbate, tocopherol acetate, butylated hydroxy benzoic acid, 6-hydroxy-2,5,7,8-tetramethylchroman-2-carboxylic acid, gallic acid, sorbic acid, lipoic acid, N,N-diethylhydroxylamine, amino-guanidine, a sulfhydryl compoun, glutathione), dihydroxy fumaric acid, lycine pidolate, arginine pilolate, nordihydroguaiaretic acid, bioflavonoids, curcumin, lysine, methionine, proline, superoxide dismutase, silymarin, tea extract, grape skin extract, grape seed extract, melanin and rosemary extract;
xxvii. Dihydroxyacetone;
xxviii. insoluble inorganic compounds selected from the group consisting of titanium dioxide, zinc oxide, zirconium oxide, iron oxide, silicone oxide, talc, carbon, calcium, magnesium hypochlorite and metallic Silver;
xxix. plant seed meal selected from the group consisting of strawberry seeds, raspberry seeds, apricot seeds, sweet almond seeds and cranberry seeds; and
xxx. oil comprising fatty acids selected from the group consisting of an unsaturated polyunsaturated fatty acid, a polyunsaturated fatty acid, an omega-3 fatty acid, an omega-6 fatty acid, linoleic and linolenic acid, gamma-linoleic acid and eicosapentaenoic acid
and derivatives, isomers and analogs thereof.

21. The composition of claim 19, wherein the composition comprises at least two active agents.

22. The composition of claim 21, wherein each of the at least two active agents exerts its therapeutic effect through at different mode of action.

23. The composition of claim 1, wherein the film forming agent is selected from the group consisting of (1) hydrophobic, high-molecular-weight, carboxylated acrylic copolymers; (2) anionic hydrophobically modified alkali-soluble acrylic polymers; and (3) acrylates/alkyl acrylate crosspolymers;
and wherein the active agent is semi-liquid or liquid at ambient temperature.

24. The composition of claim 23, wherein the active agent is selected from the group consisting of diethyltoluamide, diethyl phthalate, a sunscreen agent and permethrin.

25. The composition of claim 1, wherein the concentration of film forming agent is sufficient to elevate skin hydration, in use, in a human subject for at least 1 hour.

26. The composition of claim 1, wherein the concentration of film forming agent is sufficient to elevate skin availability of an active agent, in use.

27. The composition of claim 1, wherein the propellant contains at least one compressed gas selected form the group of (1) volatile hydrocarbons; and (2) fluorocarbon gases.

28. The composition of claim 1, wherein the propellant contains at least one compressed gas selected form the group of:
i. hydrocarbons selected from the group consisting of butane, propane and isobutene;
ii. fluorocarbon gases selected from the group consisting of 1,1,difluoro ethane, 1,1,1,2 tetrafluorethane, 1,1,1,3,3,3 hexafluoropropane and 1,1,1,2,3,3,3 heptafluoropropane.

29. A method of treating, alleviating or preventing a disorder, including:
administering topically to a surface having the disorder, a foamed composition including:
(1) about 6% to about 70% by weight of an organic carrier;
(2) about 0.1% to about 5% by weight of a surface-active agent;
(3) about 0.01% to about 5% by weight of a film forming agent;
(4) water;
(5) about 3% to about 25% by weight of the total composition of a liquefied or compressed gas propellant; and
(6) an active agent.

30. The method of claim 29, wherein the composition further includes about 0.1% to about 5% by weight of a foam adjuvant selected from the group consisting of a fatty alcohol having 15 or more carbons in their carbon chain; a fatty acid having 16 or more carbons in their carbon chain; fatty alcohols derived from beeswax and including a mixture of alcohols, a majority of which has at least 20 carbon atoms in their carbon chain; a fatty alcohol having an double bond; a fatty acid having an double bond; a branched fatty alcohol; a branched fatty acid; a fatty acid substituted with a hydroxyl group; cetyl alcohol; stearyl alcohol; arachidyl alcohol; behenyl alcohol; 1-triacontanol; hexadecanoic acid; stearic acid; arachidic acid; behenic acid; octacosanoic acid; 12-hydroxy stearic acid and mixtures thereof.

31. The method of claim 29, wherein the active agent is selected from the group consisting of a drug, a cosmetic active agent, an anti-infective, an antibiotic, an antibacterial agent, a disinfectant, an antifungal agent, an antiviral agent, an antiparasitic agent, an anti-inflammatory agent, an anti-allergic agent, a steroid, a steroidal anti-inflammatory agent, a nonsteroidal anti-inflammatory drug, an immunosuppressive agent, an immunomodulator, an immunoregulating agent, a hormone, a keratolytically active agent, an alpha hydroxyl acid, a beta-hydroxy acid, vitamin A, a vitamin A derivative, a retinoid, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a burn healing agent, an anesthetic, a protein, a peptide, a neuropeptide, a allergen, an immunogenic substance, a haptene, an oxidizing agent, an antioxidant, a dicarboxylic acid, an antiproliferative agent, an anticancer agent, a photodynamic therapy agent, a sunscreen agent, an anti-wrinkle agent, a radical scavenger, a metal oxide, an anti wrinkle agent, an anti-acne agent, a skin whitening agent, a self tanning agent, an anti-cellulite agent, a skin protective agent, a masking agent, an anti-wart agent, a refatting agent, a lubricating agent and mixtures thereof at any proportion.

32. The method of claim 29, wherein the disorder is selected from the group of insect infestation and insect bite; and wherein active agent is selected from the group consisting of permethrin, pyrethrum extract, piperonyl butoxide, diethyl toluamide and dimethyl phthalate

33. The method of claim 29, wherein the disorder is selected from the sunburn and a skin disiease, which results from UV light radiation; and wherein active agent is a sunscreen agent

34. The method of claim 29, wherein the disorder is a skin disordser which involves skin dryness as a primary etiological factor; and wherein active agent comprises at least two agents, selected from the group consisting of avocado oil, a silicone oil, cyclomethicone, dimethicone, aloe vera extract, sodium hyaluronate, licorice extract, alpha bisabolol, tocopheryl acetate and allantoin.
